(19) United States
(12) Patent Application Publication
(10) Pub. No.: US 2006/0058675 A1
(43) Pub. Date: Mar. 16, 2006

(54) Three dimensional atrium-ventricle plane detection

(75) Inventor: Bjorn Olstad, Stathelle (NO)

Correspondence Address: MCANDREWS HELD & MALLOY, LTD, 500 WEST MADISON STREET, SUITE 3400, CHICAGO, IL, 60661, US

(73) Assignee: General Electric Company

(21) Appl. No.: 11/082,296

(22) Filed: Mar. 17, 2005

Related U.S. Application Data
(60) Provisional application No. 60/606,041, filed on Aug. 31, 2004.

(51) Int. Cl. A61B 8/02

(52) U.S. Cl. 600/450

(57) ABSTRACT

The present invention relates to a method and apparatus for generating at least a 3D image responsive to moving cardiac structure and blood, and extracting clinically relevant information based on anatomical landmarks located within the heart. One embodiment of the present invention comprises at least a front end and at least one processor. The front-end is arranged to transmit ultrasound waves into the moving cardiac structure and blood of a heart and generate received signals in response to ultrasound waves backscattered from the said moving cardiac structure and blood. The at least one processor responsive to the received signals to acquire 3D ultrasound data containing at least one view of the heart, identify an AV-plane using the at least one acquired view, and generate a cardiac 3D image of at least a portion of the heart using at least one identified AV-plane. At least the 3D image may be displayed to a user.

### RELATED APPLICATIONS/INCORPORATION BY REFERENCE

This application is related to, and claims benefit of and priority from, Provisional Application No. 60/606,041, filed Aug. 31, 2004, titled “THREE DIMENSIONAL ATRIUM-VENTRICLE PLANE DETECTION”, the complete subject matter of which is incorporated herein by reference in its entirety.

complete subject matter of each of the following U.S. patent applications is incorporated by reference herein in their entirety:
U.S. patent application Ser. No. 10/248,090 filed on Dec. 17, 2002.
U.S. patent application Ser. No. 10/064,032 filed on Jun. 4, 2002.
U.S. patent application Ser. No. 10/064,083 filed on Jun. 10, 2002.
U.S. patent application Ser. No. 10/064,033 filed on Jun. 4, 2002.
U.S. patent application Ser. No. 10/064,084 filed on Jun. 10, 2002.
U.S. patent application Ser. No. 10/064,085 filed on Jun. 10, 2002.
U.S. Provisional Patent Application Ser. No. 60,605,939 (Attorney Docket Number 15-DS-00552) filed on Aug. 31, 2004.
U.S. Provisional Patent Application Ser. No. 60/605,953 (Attorney Docket Number 15-DS-00543) filed on Aug. 31, 2004.

### FEDERALLY SPONSORED RESEARCH OR DEVELOPMENT

[Not Applicable]

### BACKGROUND OF THE INVENTION

Embodiments of the present invention relate to an ultrasound system for detecting a three-dimensional (3D) atrium-ventricle plane (AV-plane). More specifically, embodiments of the present invention relate to an ultrasound system for imaging a heart, identifying an AV-plane of the heart and forming a cardiac 3D image of at least a portion of the heart using at least the AV-plane.

Echocardiography is a branch of the ultrasound field that is currently a mixture of subjective image assessment and extraction of key quantitative parameters. Evaluation of cardiac function has been hampered by a lack of well-established parameters that may be used to increase the accuracy and objectivity in the assessment of diseases, coronary artery diseases for example. It has been shown that inter-observer variability between echo-centers is unacceptably high due to the subjective nature of the cardiac motion assessment.

Research has focused on this problem, aimed at defining and validating quantitative parameters. Encouraging clinical validation studies have been reported which indicate a set of new potential parameters that may be used to increase objectivity and accuracy in the diagnosis of, for instance, coronary artery diseases. Many of the new parameters have been difficult or impossible to assess directly by visual inspection of the ultrasound images generated in real-time. The quantification has typically required a post-processing step with tedious, manual analysis to extract the necessary parameters. Determination of the location of anatomical landmarks in the heart is no exception. Time intensive post-processing techniques or complex, computation-intensive real-time techniques are undesirable.

One method disclosed in U.S. Pat. No. 5,601,084 to Sheehan et al. describes imaging and three-dimensionally modeling portions of the heart using imaging data. Another method disclosed in U.S. Pat. No. 6,099,471 to Torp et al. describes calculating and displaying strain velocity in real time. Still another method disclosed in U.S. Pat. No. 5,515,856 to Olstad et al. describes generating anatomical M-mode displays for investigations of living biological structures, such as heart function, during movement of the structure. Yet another method disclosed in U.S. Pat. No. 6,019,724 to Gronningsaeter et al. describes generating quasi-real-time feedback for the purpose of guiding procedures by means of ultrasound imaging.

### BRIEF SUMMARY OF THE INVENTION

An embodiment of the present invention relates to an ultrasound system for detecting a three-dimensional (3D) AV-plane. More specifically, an embodiment of the present invention relates to an ultrasound system for imaging a heart, identifying an AV-plane of the heart and forming a cardiac 3D image of at least a portion of the heart using at least the AV-plane.

One embodiment of the present invention relates to a system and method for generating an image responsive to moving cardiac structure and blood. One or more embodiments of the present invention relates to a an ultrasound machine adapted to generate an image responsive to moving cardiac structure and blood. This embodiment of the method comprise acquiring 3D ultrasound data containing at least one view of the moving cardiac structure and blood and identifying an AV-plane using the at least one acquired view. The method further comprises generating a cardiac 3D image using at least the identified AV-plane.

Another embodiment of the present invention relates to an ultrasound machine adapted to generate an image responsive to moving cardiac structure and blood of a heart. In this embodiment, the method comprises scanning the heart to acquire 3D ultrasound data containing at least one apical image and identifying an AV-plane using the at least one acquired apical image. At least one anatomical landmark is formed using at least the identified AV-plane and a cardiac 3D image of at least a portion of the heart if generated and displayed using at least the one anatomical landmark.

One embodiment of the present invention relates to at least a front end and at least one processor. The front-end is arranged to transmit ultrasound waves into the moving cardiac structure and blood of a heart and generate received signals in response to ultrasound waves backscattered from the moving cardiac structure and blood. The at least one processor responsive to the received signals acquires 3D ultrasound data containing at least one view of the heart, identifies an AV-plane using the at least one acquired view, and generates a cardiac 3D image of at least a portion of the heart using at least one identified AV-plane. At least the 3D image may be displayed to a user.

Certain embodiments of the present invention afford an approach to extract certain clinically relevant information from a heart after automatically locating key anatomical landmarks of the heart, such as the apex and the AV-plane.

### BRIEF DESCRIPTION OF THE DRAWINGS

FIG. 1 illustrates a block diagram of an embodiment of an ultrasound machine made in accordance with various embodiments of the present invention.

FIGS. 2A and 2B illustrate flowcharts illustrating an embodiment of a method performed by the machine shown in FIG. 1, in accordance with various embodiments of the present invention.

FIG. 3 illustrates using the method of FIGS. 2A and 2B to identify the lower parts of the basal segments and mid segments within a heart in accordance with an embodiment of the present invention.

FIG. 4 illustrates using the method of FIGS. 2A and 2B to identify a single myocardial segment or multiple myocardial segments within a heart in accordance with an embodiment of the present invention.

FIG. 5 illustrates the relationship between strain computed from strain rate imaging and strain visualized and computed from tissue motion imaging in accordance with an embodiment of the present invention.

FIG. 6 illustrates using the method of FIGS. 2A and 2B to localize a number of short axis anatomical M-modes with respect to anatomical landmarks in accordance with an embodiment of the present invention.

FIG. 7 illustrates using the method of FIGS. 2A and 2B to preset two longitudinal M-modes through two AV-plane locations in accordance with an embodiment of the present invention.

FIG. 8 illustrates using the method of FIGS. 2A and 2B to preset a curved M-mode within a myocardial segment from the apex and down to the AV-plane in accordance with an embodiment of the present invention.

FIG. 9 illustrates using the method of FIGS. 2A and 2B to preset a Doppler sample volume relative to detected anatomical landmarks in accordance with an embodiment of the present invention.

FIG. 10 illustrates using the method of FIGS. 2A and 2B to define a set of points within myocardial segments to perform edge detection in accordance with an embodiment of the present invention.

FIG. 11 illustrates using the method of FIGS. 2A and 2B to differentiate between two chambers of a heart in accordance with an embodiment of the present invention.

FIG. 12 illustrates using the method of FIGS. 2A and 2B to tag a display of a heart with a grid and track the grid in accordance with an embodiment of the present invention.

FIG. 13 illustrates using the method of FIGS. 2A and 2B to acquire and display key parameter information in accordance with an embodiment of the present invention.

FIG. 14 illustrates using the method of FIGS. 2A and 2B to create and display key parameter information acquired using a method similar to that of FIG. 13 in accordance with one embodiment of the present invention.

FIG. 15 illustrates using the method of FIGS. 2A and 2B to display a 3D geometrical model of a least a portion of the heart in accordance with one embodiment of the present invention.

The foregoing summary, as well as the following detailed description of certain embodiments of the present invention, will be better understood when read in conjunction with the appended drawings. It should be understood, however, that the present invention is not limited to the arrangements and instrumentality shown in the attached drawings.

### DETAILED DESCRIPTION OF THE INVENTION

An embodiment of the present invention relates to an ultrasound system for detecting a 3D AV-plane. More specifically, an embodiment of the present invention relates to an ultrasound system for imaging a heart, identifying at least an AV-plane of the heart and forming a cardiac three-dimensional 3D image of at least a portion of the heart using at least the AV-plane. Moving cardiac structure is monitored to accomplish this function. As used herein, the term structure comprises non-liquid and non-gas matter, such as cardiac tissue for example. An embodiment of the present invention provides improved, real-time visualization and quantative assessment of certain clinically relevant or key parameters of the heart. The moving structure is characterized by a set of analytic or key parameter values corresponding to anatomical points within a myocardial segment of the heart. The set of analytic or key parameter values may comprise, for example, tissue velocity values, time-integrated tissue velocity values, B-mode tissue intensity values, tissue strain rate values, blood flow values, and mitral valve inferred values.

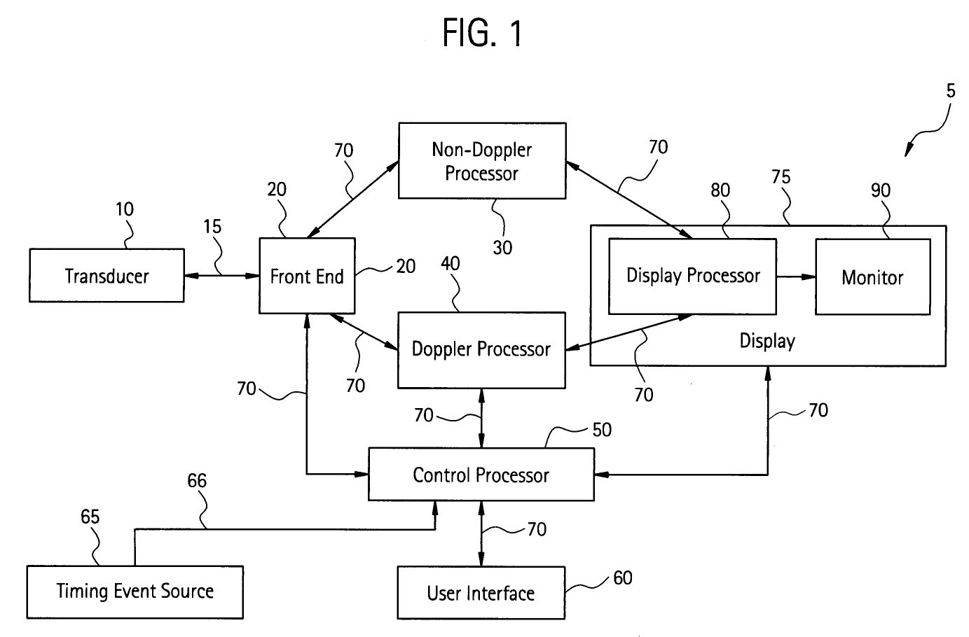

FIG. 1 illustrates an embodiment of an ultrasound machine, generally designated 5, in accordance with embodiments of the present invention. A transducer 10 transmits ultrasound waves into a subject by converting electrical analog signals to ultrasonic energy and receives the ultrasound waves backscattered from the subject by converting ultrasonic energy to analog electrical signals. A front-end 20, that in one embodiment comprises a receiver, transmitter, and beamformer, may be used to create the necessary transmitted waveforms, beam patterns, receiver filtering techniques, and demodulation schemes that are used for the various imaging modes. Front-end 20 performs such functions, converting digital data to analog data and vice versa. Front-end 20 interfaces to transducer 10 using analog interface 15 and interfaces to a non-Doppler processor 30, a Doppler processor 40 and a control processor 50 over a bus 70 (digital bus for example). Bus 70 may comprise several digital sub-buses, each sub-bus having its own unique configuration and providing digital data interfaces to various parts of the ultrasound machine 5.

Non-Doppler processor 30 is, in one embodiment, adapted to provide amplitude detection functions and data compression functions used for imaging modes such as B-mode, M-mode, and harmonic imaging. Doppler processor 40, in one embodiment provides clutter filtering functions and movement parameter estimation functions used for imaging modes such as tissue velocity imaging (TVI), strain rate imaging (SRI), and color M-mode. In one embodiment, the two processors, 30 and 40, accept digital signal data from the front-end 20, process the digital signal data into estimated parameter values, and passes the estimated parameter values to processor 50 and a display 75 over digital bus 70. The estimated parameter values may be created using the received signals in frequency bands centered at the fundamental, harmonics, or sub-harmonics of the transmitted signals in a manner known to those skilled in the art.

Display 75 is adapted, in one embodiment, to provide scan-conversion functions, color mapping functions, and tissue/flow arbitration functions, performed by a display processor 80 which accepts digital parameter values from processors 30, 40, and 50, processes, maps, and formats the digital data for display, converts the digital display data to analog display signals, and communicate the analog display signals to a monitor 90. Monitor 90 accepts the analog display signals from display processor 80 and displays the resultant image.

A user interface 60 enables user commands to be input by the operator to the ultrasound machine 5 through control processor 50. User interface 60 may comprise a keyboard, mouse, switches, knobs, buttons, track balls, foot pedals, voice control and on-screen menus, among other devices.

A timing event source 65 generates a cardiac timing event signal 66 that represents the cardiac waveform of the subject. The timing event signal 66 is input to ultrasound machine 5 through control processor 50.

In one embodiment, control processor 50 comprises the central processor of the ultrasound machine 5, interfacing to various other parts of the ultrasound machine 5 through digital bus 70. Control processor 50 executes the various data algorithms and functions for the various imaging and diagnostic modes. Digital data and commands may be communicated between control processor 50 and other various parts of the ultrasound machine 5. As an alternative, the functions performed by control processor 50 may be performed by multiple processors, or may be integrated into processors 30, 40, or 80, or any combination thereof. As a further alternative, the functions of processors 30, 40, 50, and 80 may be integrated into a single PC backend.

Once certain anatomical landmarks of the heart are identified, (e.g., the AV-planes and apex as described in U.S. patent application Ser. No. 10/248,090 filed on Dec. 17, 2002) certain relevant information (one or more key parameters for example) may be extracted and displayed to a user of the ultrasound machine 5 (on a display for example) in accordance with various aspects of the present invention. The various processors of the ultrasound machine 5 described above may be used to extract and display relevant information from various locations within the heart.

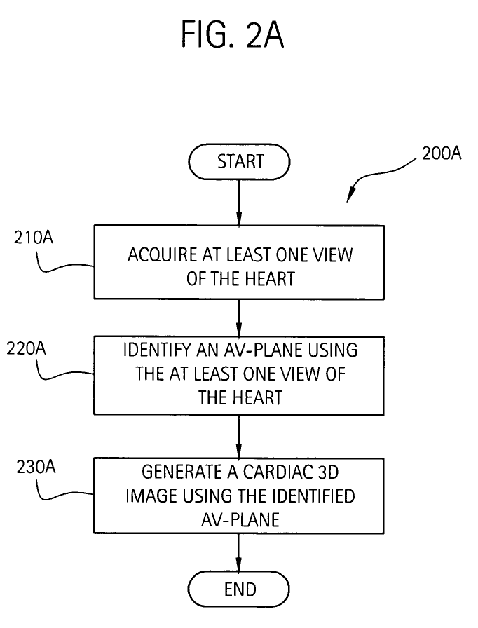

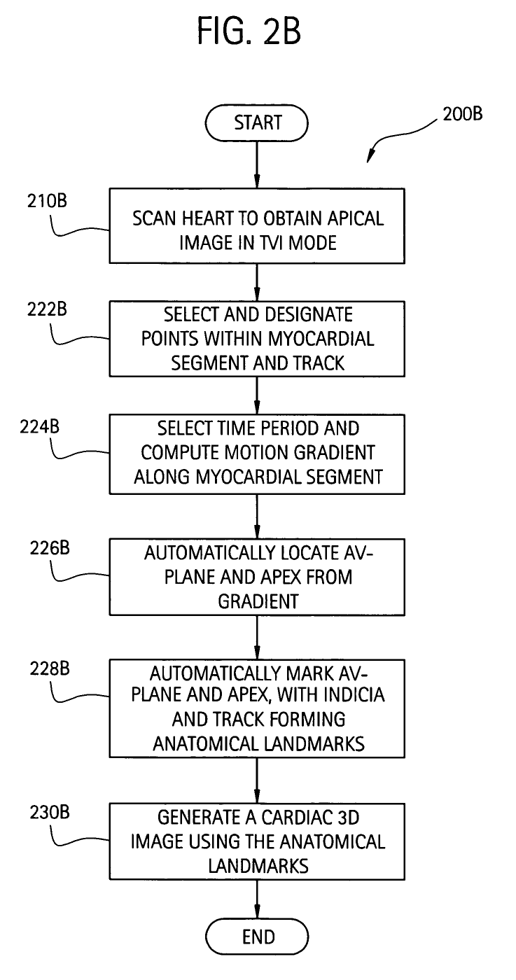

One embodiment of the present invention relates to acquiring at least one view of the heart and forming a cardiac 3D image of the AV-plane of the heart, performing real-time visualization and quantative assessment of certain key parameters of the heart. More specifically, one embodiment of the present invention may be used to generate 3D images of one or more valves (the mitral valve for example) and the surrounding structure. FIG. 2A depicts a high level flow chart illustrating a method 200A for generating a cardiac 3D image used to perform real-time visualization and quantative assessment of certain key parameters of the heart. In the illustrated embodiment, the method 200A comprises Step 210A, acquiring at least one view of the heart while imaging the heart. Step 220A comprises identifying an AV-plane of the heart while imaging the heart using the at least one acquired view. Step 230A comprises generating a cardiac 3D image (automatically in one embodiment) using the identified AV-plane.

FIG. 2B depicts a flow chart illustrating an embodiment of a method 200B (similar to method 200A of FIG. 2A) performed using a machine 5 illustrated in FIG. 1 for example in accordance with various embodiments of the present invention. Method 200B comprises Step 210B, scanning the heart to obtain at least one apical image of the heart (in TVI mode for example). Step 222B comprises selecting and designating one or more points within the myocardial segment of the heart and tracking the selected and designated points.

One embodiment of method 200 further comprises Step 224B, selecting a time period and computing one or more motion gradients along at least one myocardial segment. Step 226B comprises locating an AV-plane and apex (automatically for example) using at least one of the gradients computed in Step 224B for example.

Method 200B further comprises Step 228B, automatically marking the AV-plane and apex with indicia and tracking the marked AV-plane and apex forming at least one anatomical landmark. Step 230B comprises generating at least one cardiac 3D image using at least one of the anatomical landmarks formed in Step 228B.

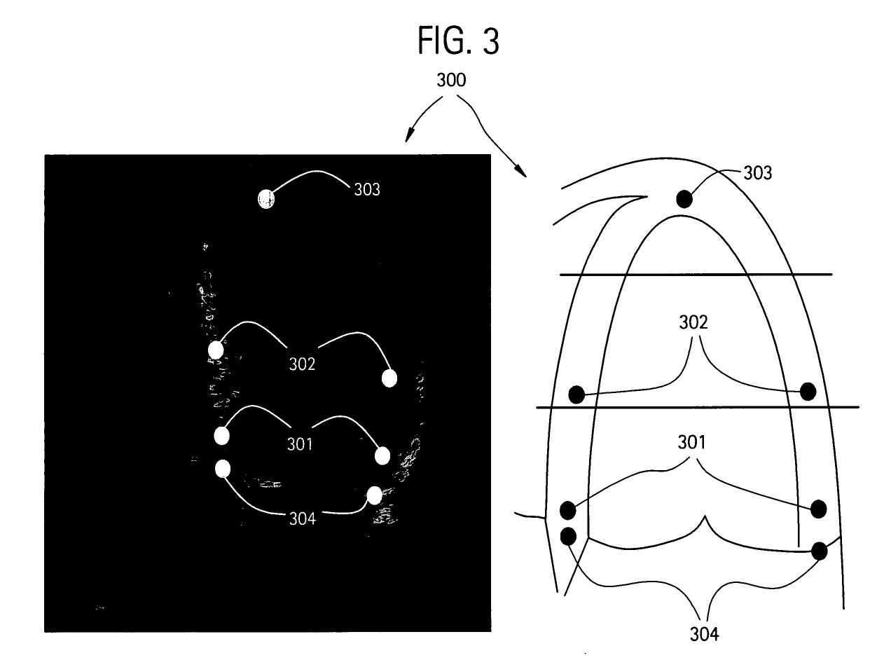

FIG. 3 depicts a diagram using methods 200A and 200B illustrated in FIGS. 2A and 2B respectively, to identify at least the lower parts of the basal and mid segments within a heart in accordance with at least one embodiment of the present invention. Detected landmarks may be used to identify locations within the heart provided by relative positioning and local image characteristics. FIG. 3 illustrates two depictions of a heart 300. An image of the heart 300 with various markers overlaying certain anatomical locations is shown on the left of FIG. 3. A graphical illustration of the heart 300 with various markers overlaying certain anatomical locations is shown on the right of FIG. 3. FIG. 3 further provides an example in which the lower parts of the myocardium in the basal segments 301 of the heart 300 and the lower part of the mid segments 302 of the heart 300 are identified relative to the detected landmarks (i.e., apex 303 and AV-plane 304).

Once certain anatomical landmarks of the heart are identified, (e.g., the AV-planes and apex as described in U.S. patent aplication Ser. No. 10/248,090 filed on Dec. 17, 2002) certain clinically relevant information may be extracted and displayed to a user of the ultrasound system 5 in accordance with various aspects of the present invention. The various processors of the ultrasound machine 5 described above may be used to extract and display information from various locations within the heart.

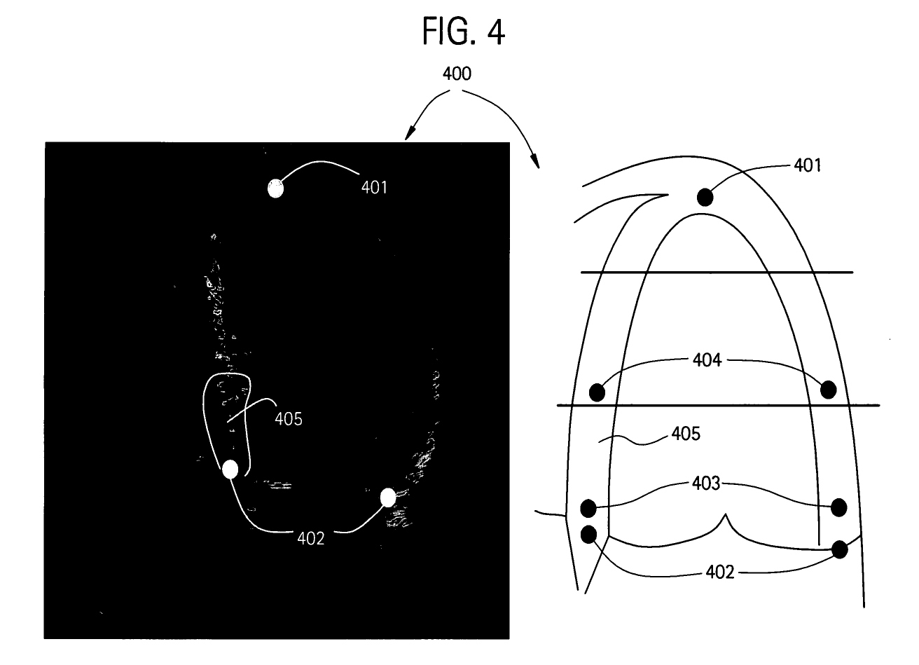

FIG. 4 depicts a diagram illustrating using methods 200A and 200B of FIGS. 2A and 2B to identify single or multiple myocardial segments within a heart and extract information, in accordance with at least one embodiment of the present invention. FIG. 4 illustrates how locations in the heart 400 (similar to those shown in FIG. 3) combined with boundary detection, may be used to identify a single myocardial segment 405 or multiple myocardial segments. In one embodiment, the locations are marked as apex 401, AV-plane 402, lower part of basal segments 403, and lower part of mid segments 404. It is contemplated that segments defined in the 16-segment model of ASE or other similar schemes may be identified. Based on such segmentation, representative key parameters may be computed for the segment 405 in accordance with various aspects of the present invention.

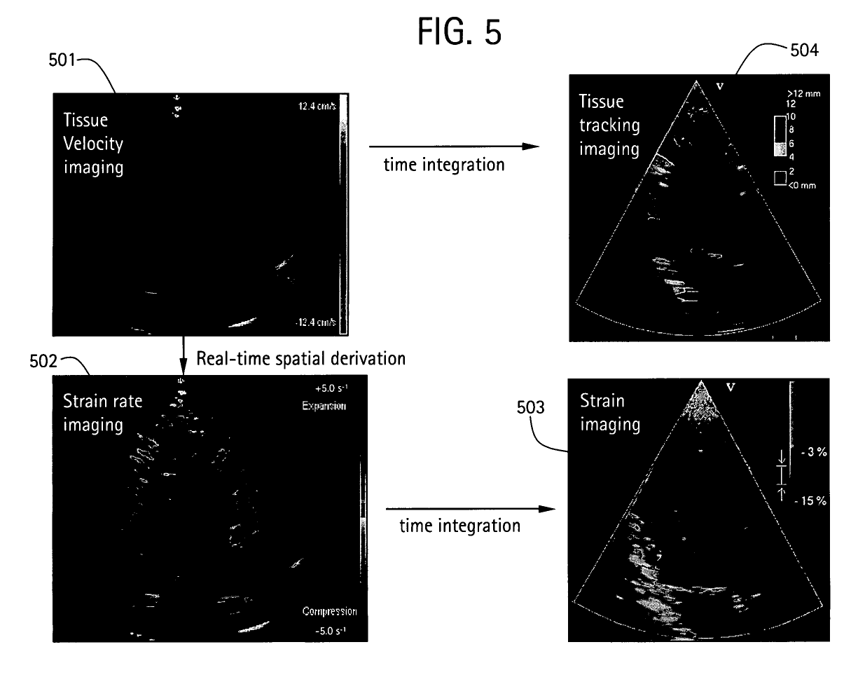

FIG. 5 depicts a diagram illustrating the relationship between strain computed from strain rate imaging and strain visualized and computed from tissue motion imaging in accordance with an embodiment of the present invention. Tissue velocity image 501 is illustrated in the upper left of FIG. 5. It is contemplated that, if the gradient of the tissue velocity is computed along the ultrasound beam, a strain rate image 502 may be obtained. One example of such strain rate image is shown in the lower left of FIG. 5. The strain rate values for a given spatial or anatomical location may be combined for a time interval (such as systole for example) to compute the local strain as a total deformation in percentage: the lower right of FIG. 5 illustrates such an example in which the total systolic strain 503 is used to color encode myocardium. Alternatively, discrete color encoding 504 of the systolic motion values may be constructed as shown in the upper right corner of FIG. 5. It is contemplated that all these data sources represent possible quantitative clinically relevant information that may be extracted either as simple values or time profiles at locations relative to the detected landmarks.

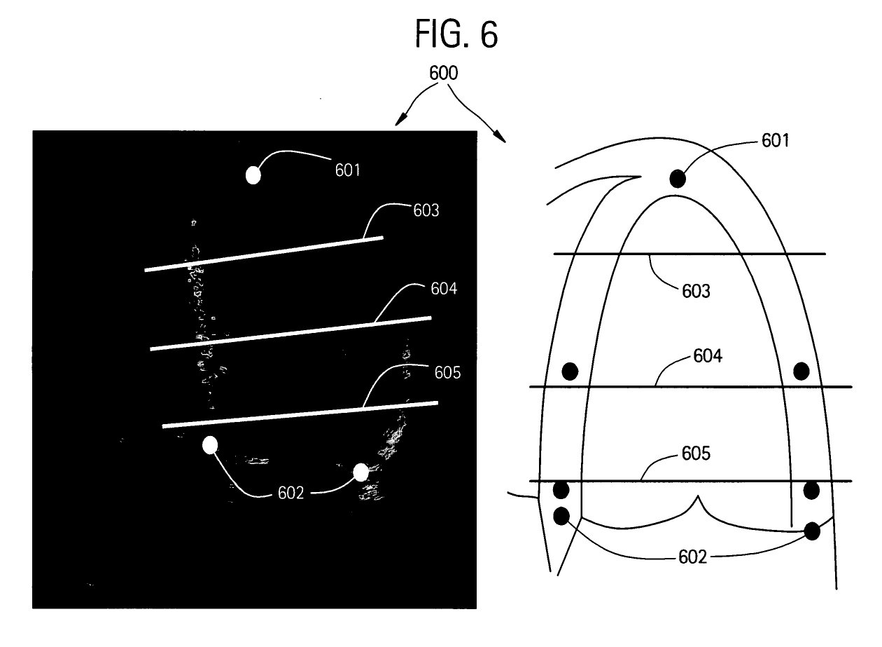

The detected landmarks and related locations may be used to preset the spatial location for acquisition or extraction of information. FIG. 6 depicts a diagram that illustrates using methods 200A and 200B of FIGS. 2A and 2B to localize a number of short axis anatomical M-modes with respect to anatomical landmarks, extracting information in accordance with an embodiment of the present invention. FIG. 6 illustrates how a given number of short axis anatomical M-modes 603, 604, and 605 may be localized as fixed geometrical percentages relative to apex 601 and the two AV-plane locations 602 within a heart 600, in accordance with an embodiment of the present invention.

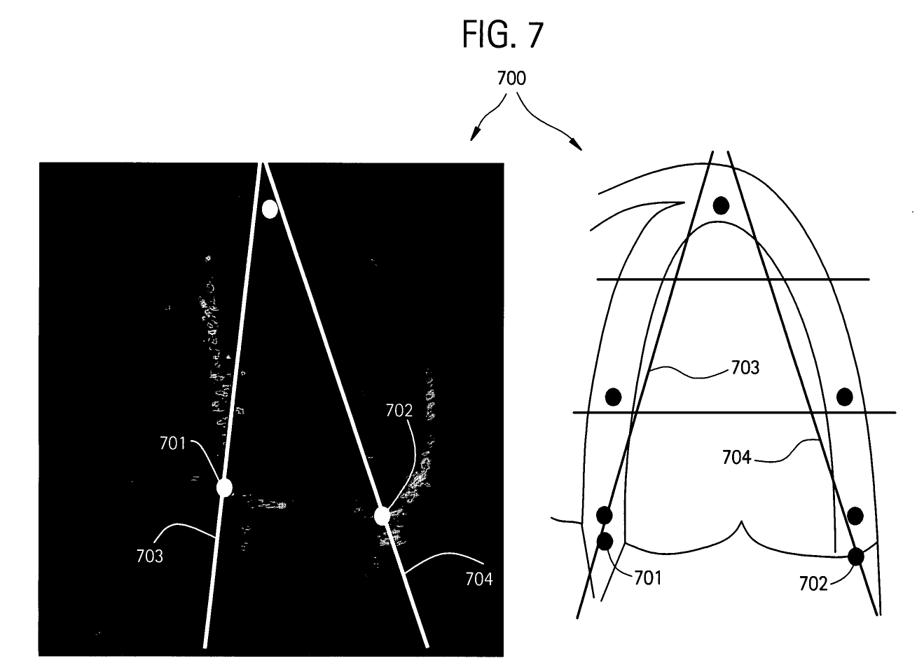

FIG. 7 depicts a diagram that illustrates using methods 200A and 200B of FIGS. 2A and 2B to preset two longitudinal M-modes through two AV-plane locations, extracting information, in accordance with an embodiment of the present invention. FIG. 7 illustrates how two longitudinal M-modes 703 and 704 may be preset through the two AV-plane locations 701 and 702 in order to display the longitudinal AV-motion in two M-modes within the heart 700, in accordance with an embodiment of the present invention.

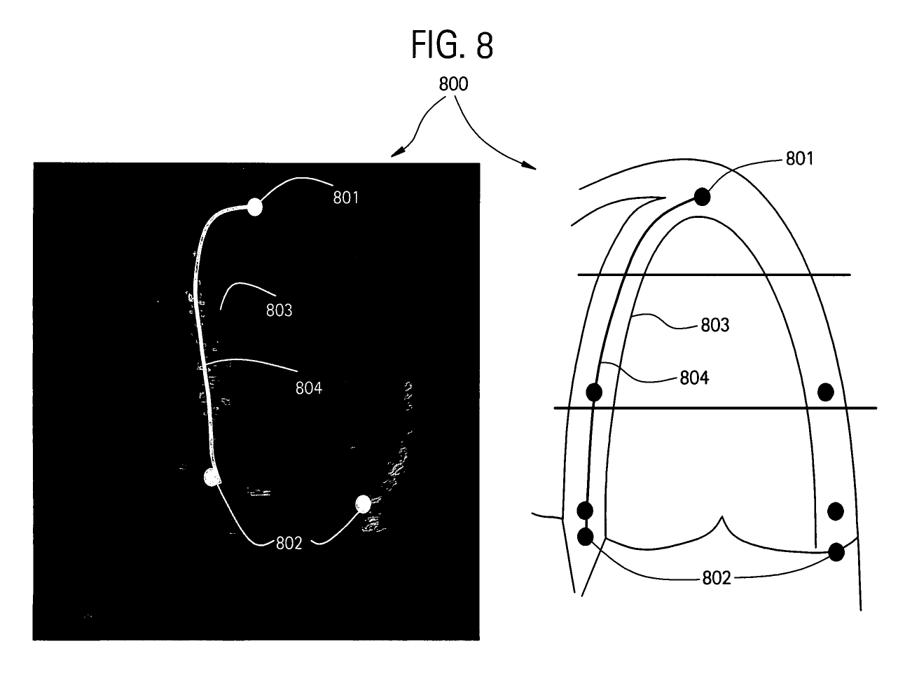

FIG. 8 depicts a diagram illustrating using methods 200A and 200B of FIGS. 2A and 2B to preset a curved M-mode within a myocardial segment from apex down to the AV-plane, extracting information, in accordance with an embodiment of the present invention. FIG. 8 illustrates how a curved M-mode 804 from apex 801 down to the AV-plane 802 in the middle of myocardium 803 may be preset using the landmarks alone or in combination with local image analysis to keep the curve 804 inside myocardium 803 within the heart 800, in accordance with an embodiment of the present invention.

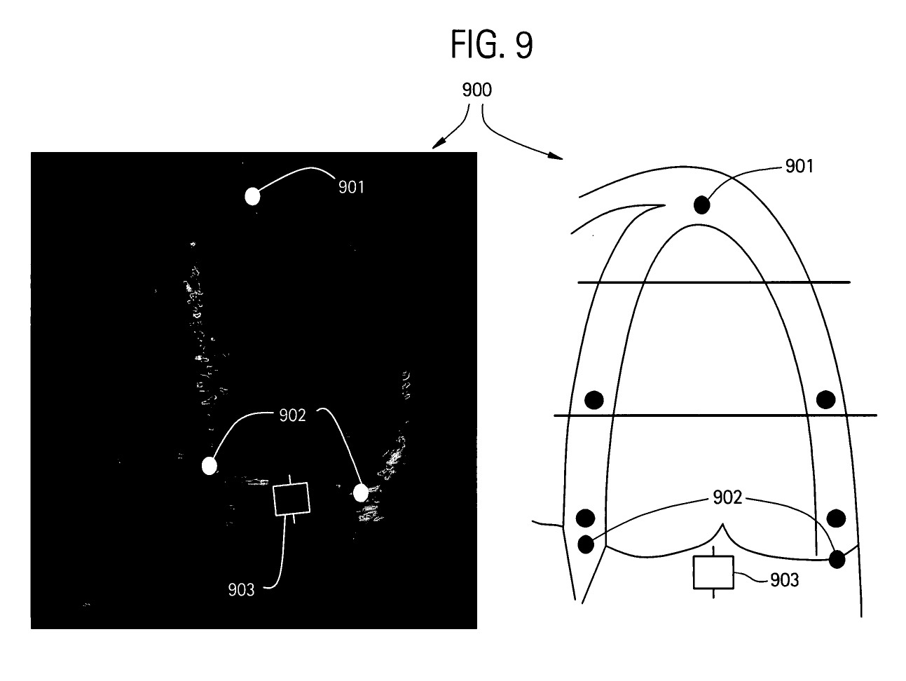

FIG. 9 depicts a diagram illustrating using methods 200A and 200B of FIGS. 2A and 2B to preset a Doppler sample volume relative to detected anatomical landmarks, extracting information, in accordance with an embodiment of the present invention. FIG. 9 illustrates how a sample volume 903 for Doppler measurements may be preset relative to the detected landmarks 901 (apex) and 902 (AV-plane) within the heart 900. Such a technique may be applied to PW and CW Doppler, for inspection of blood flow and measurement of myocardial function.

In accordance with at least one embodiment of the present invention, a region-of-interest (ROI) may be preset with respect to the anatomical landmarks extracting information from these clinically relevant locations. The extracted information may include one or more of Doppler information over time, velocity information over time, strain rate information over time, strain information over time, M-mode information, deformation information, displacement information, and B-mode information.

The locations of the M-modes, curved M-modes, sample volumes, and ROI's may be tracked in order to follow the motion of the locations, in accordance with an embodiment of the present invention. Further, indicia may be overlaid onto the anatomical landmarks and/or the clinically relevant locations to clearly display the positions of the landmarks and/or locations.

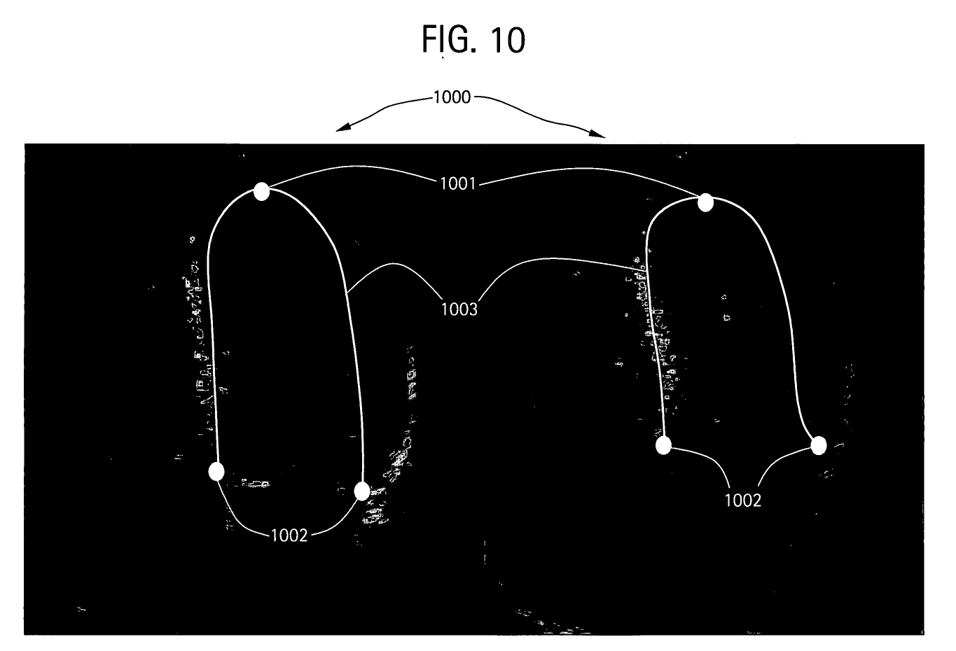

FIG. 10 depicts a diagram illustrating using methods 200A and 200B of FIGS. 2A and 2B to define a set of points within myocardial segments performing edge detection to extract information about the associated endocardium, in accordance with an embodiment of the present invention. Automatic edge detection of the endocardium remains a challenging task. FIG. 10 illustrates how the techniques discussed herein (i.e., similar to the curved M-mode localization) may be used to either define a good ROI for the edge detection, or provide an initial estimate that may be used to search for the actual boundary with edge detection algorithms such as active contours. FIG. 10 illustrates two views of a heart 1000 identifying the apex 1001 and the AV-plane 1002. A contour 1003, estimating the approximate inside of myocardial segments in the heart 1000 based on the anatomical landmarks, is drawn as the apex and AV-plane locations are tracked. Edge detection of the endocardium may then be performed using edge detection techniques using the contour as a set of starting points.

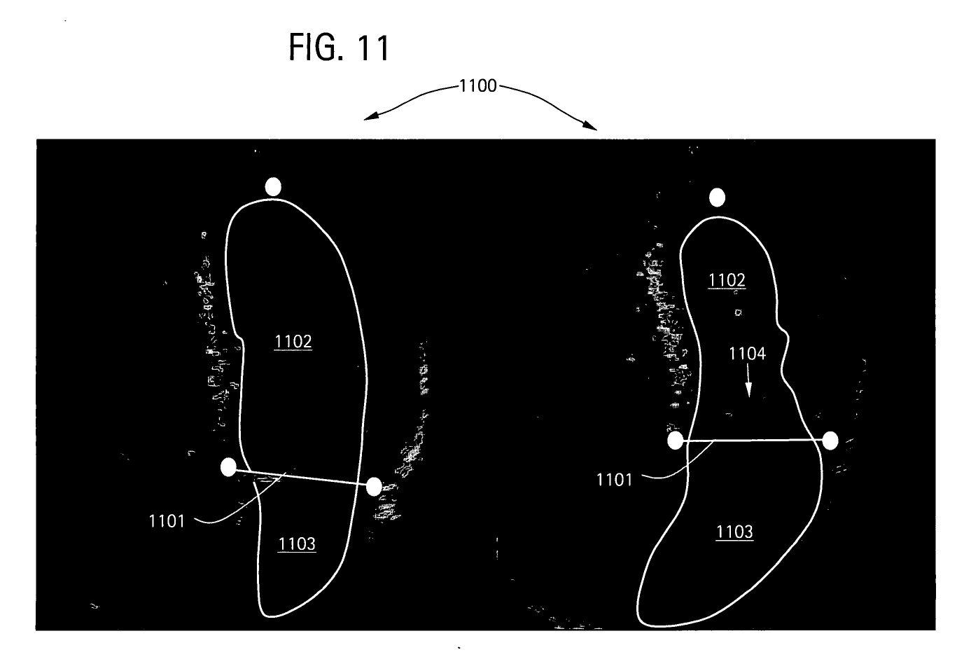

FIG. 11 depicts a diagram illustrating using methods 200A and 200B of FIGS. 2A and 2B to differentiate between two chambers of a heart and to extract information, in accordance with an embodiment of the present invention. FIG. 11 shows a different application in edge detection within two views of a heart 1100. Even an ideal blood/tissue segmentation may not, at all instances in the cardiac cycle, be able to separate between the ventricle 1102 and the atrium 1103. The two chambers 1102 and 1103 are completely connected with blood in diastole when the mitral valve 1104 is open. Detection of the AV-plane 1101 may be used to separate a blood/tissue segmentation into the ventricle and atrial components.

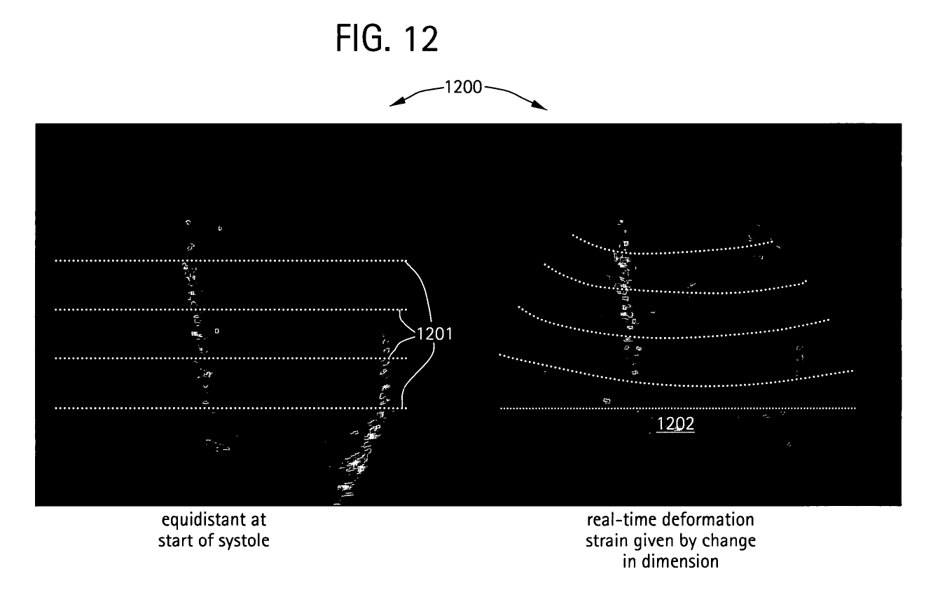

FIG. 12 depicts a diagram illustrating using methods 200A and 200B of FIGS. 2A and 2B to tag a display of a heart with a grid and track the grid to extract information, in accordance with an embodiment of the present invention. FIG. 12 illustrates one method for implementing tagging display based on tissue tracking. In accordance with an embodiment of the present invention, a time interval relative to the cardiac cycle is selected. The time interval may equal the complete cardiac cycle, for example. At the start of the time interval, a fixed graphical grid 1201 is drawn on top of the ultrasound image 1200. Any shape, including any one or two-dimensional grids may be used. The left hand side of FIG. 12 illustrates a one-dimensional grid 1201 where equidistant horizontal lines are used. It is also contemplated the equidistant set of lines with constant depth in the polar geometry representation of the ultrasound image may be used. The anatomical locations are then tracked throughout the selected time interval with either one-dimensional techniques along the ultrasound beam or two-dimensional techniques.

The right hand side of FIG. 12 illustrates the display frame in the selected time interval, wherein the motion and deformation of the original grid pattern 1201 is used to visualize the motion and strain properties. The display mode might be attractive to clinicians because it resembles tagging MR used as a gold reference for in-vivo measurements of strain. The detection of landmarks like apex and the AV-plane locations may further enhance the display mode by presetting the grid 1201 relative to the landmarks. Such presetting may assure that a grid line passes through both apex and the AV-plane. The intermediate locations may, for instance, be selected such that the displayed deformations correspond with the appropriate vascular territories. A special grid structure or band 1202 could be added around the AV-plane that corresponds to normal or expected longitudinal motion.

One embodiment of the present invention relates to acquiring a 3D image of at least a portion heart (one or more valves for example) for performing meaningful cardiac assessment. It is contemplated that the AV-plane may be used to optimize a 3D acquisition for rendering mitral valve, enabling 3D reconstruction of the mitral annulus motion for example.

One embodiment of the present invention relates to an ultrasound system for imaging a heart, identifying an AV-plane of the heart and forming a cardiac 3D image of at least a portion of the heart. More specifically, one embodiment of the present invention comprises identifying at least a mitral plane in the heart in cardiac 3D acquisition. The AV-plane may be used in such 3D acquisition to position and generate one or more optimized views/renderings of at least a heart valve (the mitral valve and neighboring structure for example).

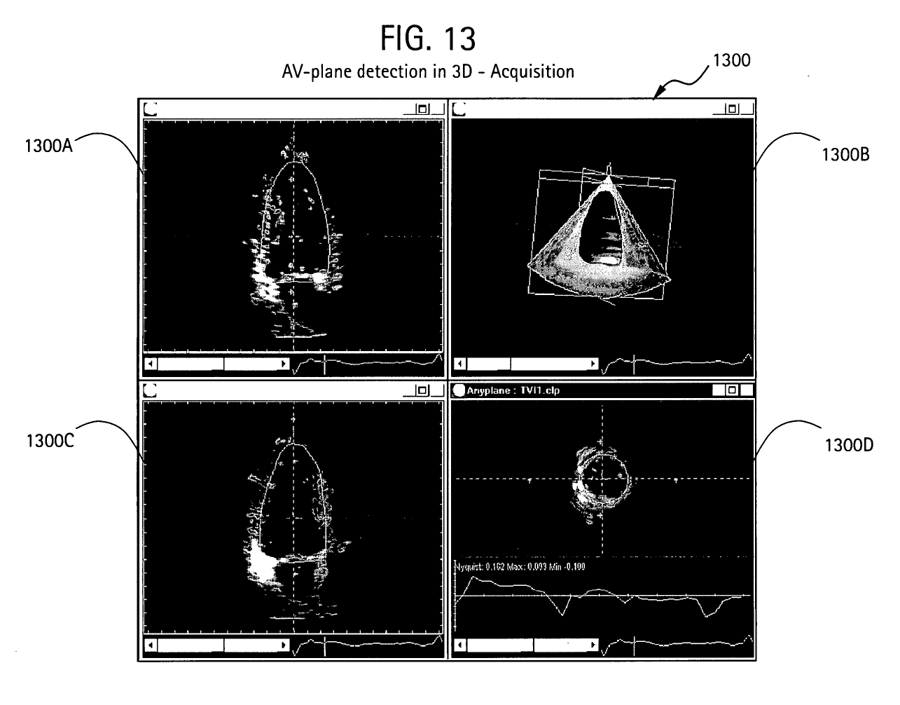

FIG. 13 illustrates one method, generally designated 1300, for acquiring and displaying key parameter information (tissue velocity for example) extracted from one or more locations in a cardiac 3D set, using the methods 200A and 200B in accordance with one or more embodiments of the present invention. It should be appreciated that the same analysis applied to 2D images as described previously may be applied to identify at least the apex and the AV-plane and automatically generating a 3D apical view. In this embodiment, four images, 1300A, 1300B, 1300C and 1300D, are generated. It should be appreciated that more, less or different views may be generated.

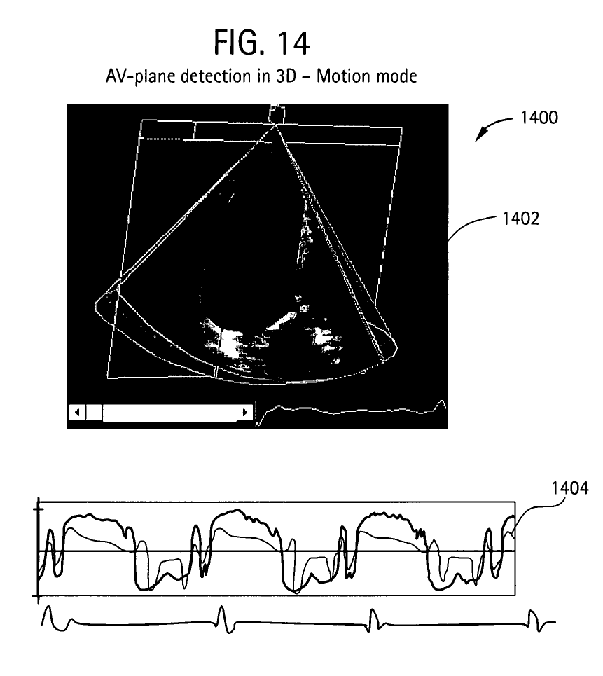

FIG. 14 depicts one method, generally designated 1400, for creating and displaying a 3D dynamic model using methods 200A and 200B in accordance with embodiments of the present invention. In this embodiment, method 1400 automatically creates and displays the 3D dynamic model using the associated key parameters (velocity values for example) extracted from one or more locations similar to that provided previously in FIG. 13. In this embodiment, method 1400 may display the key parameters (the velocity pattern) in a real-time 3D format 1402 alone or together with a post-processing, graphical format 1404.

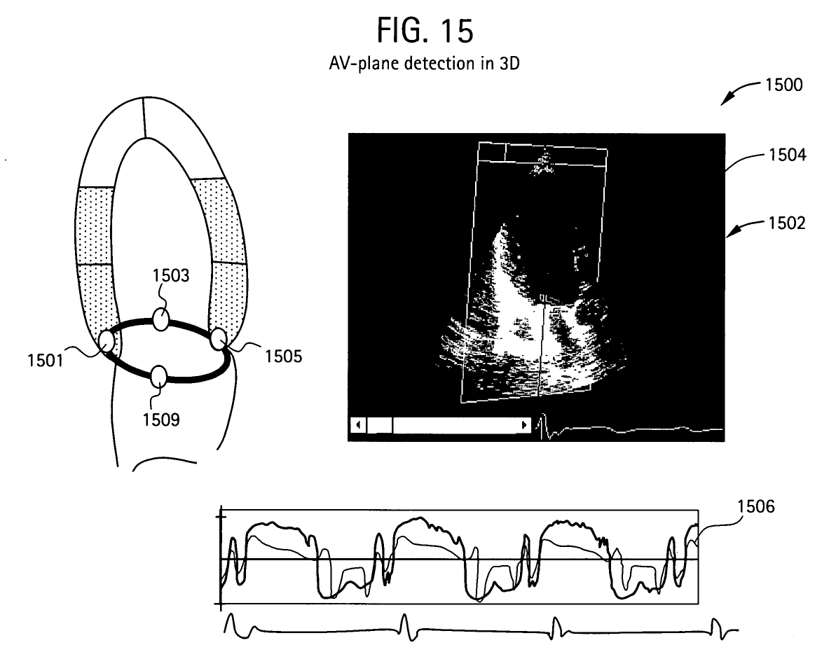

Similarly, FIG. 15 depicts a method, generally designated 1500, for displaying a geometrical model in accordance with one or more embodiments of the present invention. In the illustrated embodiment, FIG. 15 displays four AV locations 1501, 1503, 1505 and 1507 extracted from an apical chamber of the heart and a 3D reconstruction 1502 of the left ventricle and the mitral valve together with motion patterns of the mitral annulus 1504. The 3D reconstruction of the mitral annulus 1504 alone or with associated velocity patterns 1506 (including rest and peak velocities) may be automated, and the differences between the wall segments (in terms of timing and excursion) may be both graphically visualized and quantified.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. In an ultrasound machine for generating an image responsive to moving cardiac structure and blood, the method comprising:
acquiring 3D ultrasound data containing at least one view of the moving cardiac structure and blood;
identifying an AV-plane using said at least one acquired view; and
generating a cardiac 3D image using at least said identified AV-plane.

2. The method of claim 1 comprising generating a cardiac 3D image of at least a portion of the moving cardiac structure.

3. The method of claim 1 comprising displaying at least said 3D image on a display of the ultrasound machine.

4. The method of claim 1 comprising scanning the moving cardiac structure and blood to obtain at least one apical image.

5. The method of claim 1 comprising identifying at least a mitral plane of the moving cardiac structure and blood.

6. The method of claim 1 comprising displaying said 3D image in at least one of a real-time 3D format and a post-processing format.

7. The method of claim 1 comprising displaying at least a 3D reconstruction of at least one valve of the moving cardiac structure and blood.

8. The method of claim 7 further comprising displaying at least one velocity pattern associated with said at least one valve.

9. In an ultrasound machine for generating an image responsive to moving cardiac structure and blood of a heart, the method comprising:
scanning the heart to acquire 3D ultrasound data containing at least one apical image;
identifying an AV-plane using said at least one acquired apical image;
forming at least one anatomical landmark using at least said identified AV-plane; and
generating and displaying a cardiac 3D image of at least a portion of the heart using at least said one anatomical landmark.

10. The method of claim 9 comprising identifying at least a mitral plane of heart.

11. The method of claim 9 comprising displaying said 3D image in at least one of a real-time 3D format and a post-processing format.

12. The method of claim 9 comprising displaying at least a 3D reconstruction of at least a valve of the heart.

13. The method of claim 12 wherein said valve comprises a mitral valve.

14. The method of claim 13 further comprising displaying at least one velocity pattern associated with said mitral valve.

15. The method of claim 9 comprising selecting and designating points within a myocardial segment of the heart.

16. In an ultrasound machine for generating an image responsive to moving cardiac structure and blood within a heart of a subject, an apparatus comprising:
a front-end arranged to transmit ultrasound waves into the moving cardiac structure and blood and generate received signals in response to ultrasound waves backscattered from said moving cardiac structure and blood;
at least one processor responsive to said received signals acquiring at least 3D ultrasound data containing at least one view of the heart, identifying an AV-plane using said at least one acquired view, and generating a cardiac 3D image of at least a portion of the heart using at least said identified AV-plane.

17. The apparatus of claim 16 further comprising a display processor and monitor to display at least said 3D image of at least a portion of the heart.

18. The apparatus of claim 17 wherein said display processor and monitor displays at least said 3D image in at least one of a real-time 3D format and a post-processing format.

19. The apparatus of claim 16 further comprising a display processor and monitor to display at least a 3D reconstruction of at least a mitral valve of the heart.

20. The apparatus of claim 19 wherein said display processor and monitor displays at least one velocity pattern associated with said mitral valve.
